(19) United States
(12) Patent Application Publication
(10) Pub. No.: US 2006/0280710 A1
(43) Pub. Date: Dec. 14, 2006

(54) Volumizing hair compositions

(75) Inventors: Mike Wong, Plainsboro, NJ (US); Eva Zion, Bridgewater, NJ (US); Jon Lamine Toliver, North Brunswick, NJ (US); Frederic Cervantes, Westfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/147,587

(22) Filed: Jun. 8, 2005

(51) Int. Cl. A61K 8/72; A61K 8/55

(52) U.S. Cl. 424/70.11; 424/70.21; 424/70.23

(57) ABSTRACT

The present invention relates to hair compositions comprising at least one lecithin, at least one amphoteric surfactant, at least one nonionic surfactant, at least one film forming polymer, and at least one cationic polymer. The compositions are preferably used to add volume to hair while maintaining the hair's natural shape and/or its curl definition.

### BACKGROUND OF THE INVENTION

The present invention relates to hair compositions comprising at least one lecithin, at least one amphoteric surfactant, at least one nonionic surfactant, at least one film forming polymer, and at least one cationic polymer. Also preferably present in the invention compositions is water. The compositions are preferably used to maintain hair's natural shape and/or its curl definition.

People with fine or thin hair oftentimes use “volumizing” shampoos in order to add volume and body to their hair. Conventional volumizing shampoos, however, possess certain drawbacks such as, for example, a failure to provide real volume benefits due to their being too heavy on the hair, thereby weighing it down. Another drawback associated with volumizing shampoos is their inability to provide appreciable conditioning, frizz control and curl retention benefits onto hair treated therewith.

### SUMMARY OF THE INVENTION

The present inventors have now discovered compositions that address the above problems. These compositions comprise at least one lecithin, at least one amphoteric surfactant, at least one nonionic surfactant, at least one film forming polymer, and at least one cationic polymer, wherein the compositions are employed at a pH range of from 5 to 12. The first three components listed (i.e., at least one lecithin, at least one amphoteric surfactant, at least one nonionic surfactant) are sometimes referred to as a “LAN system” or simply as “LAN” herein. Preferably, the film forming polymer is a non-neutralized resin.

### DETAILED DESCRIPTION

Other than in the operating examples, or where otherwise indicated, all numbers expressing quantities of ingredients and/or reaction conditions, are to be understood as being modified in all instances by the term “about”.

Lecithins are mixtures of phospholipids, i.e., of diglycerides of fatty acids linked to an ester of phosphoric acid. Preferably, lecithins are diglycerides of stearic, palmitic, and oleic acids linked to the choline ester of phosphoric acid. Lecithin is usually defined either as pure phosphatidyl cholines or as crude mixtures of phospholipids which include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids.

The lecithin used in the present invention include the types described above and may be present in the form of a liquid, powder, or granules. Lecithins useful in the invention include, but are not limited to, soy lecithin and hydroxylated lecithin. For example, ALCOLEC S is a fluid soy lecithin, ALCOLEC F 100 is a powder soy lecithin, and ALCOLEC Z3 is a hydroxylated lecithin, all of which are available from the American Lecithin Company.

In the present invention, lecithin is preferably used in an amount greater than 0 to 10% by weight, preferably from 0.01 to 5% by weight, based on the weight of the composition as a whole. Since lecithin itself is not necessarily a pure raw material and may have free glycerides, glycerin, fatty acids, and soaps, adjustments in this amount may need to be made, i.e., one source of lecithin may require different ratios of nonionic and amphoteric surfactants than another to achieve, e.g., maximum clarity of solution. Preferably, the composition of the invention forms a clear solution, though the purpose of the invention is achieved just as effectively with a cloudy or slightly cloudy solution, etc.

A group of phospholipids which can be used in the present invention as lecithins are multifunctional biomimetic phospholipids, including, for example, the following multifunctional biomimetic phospholipids manufactured by Mona Industries: PHOSPHOLIPID PTC, PHOSPHOLIPID CDM, PHOSPHOLIPID SV, PHOSPHOLIPID GLA, and PHOSPHOLIPID EFA.

The amphoteric surfactants useful in the present invention include, but are not limited to, betaines, sultaines, hydroxysultaines, alkyl amphodiacetates, alkyl amphodipropionates, and imidazolines, or salts thereof. It is recognized that other fatty acid condensates such as those formed with amino acids, proteins, and the like are suitable. Cocamphodipropionate is particularly preferred, for example, MIRANOL C2M-SF Conc. (disodium cocamphodipropionate), in its salt-free form, available from Rhône-Poulenc. Also preferred is CROSULTAINE C-50 (cocamidopropyl hydroxysultaine), available from Croda. The amphoteric surfactants are present in the composition in an amount ranging from greater than 0 to 30% by weight, preferably from 2 to 10% by weight, based on the weight of the composition as a whole. When the lecithin/amphoteric/nonionic system is employed as a carrier for a water-insoluble polymer or resin, the amphoteric surfactants are preferably present in the composition in an amount ranging from 6 to 10% by weight. When the lecithin/amphoteric/nonionic system is employed as a carrier for a lipophilic material, the amphoteric surfactants are preferably present in the composition in an amount ranging from 4 to 8% by weight. Other amphoteric surfactants useful in the present invention include disodium wheatgermimido PEG-2 sulfosuccinate, available under the trade name MACKANATE WGD from McIntyre Group Ltd. and disodium soyamphodiacetate, available under the trade name MACKAM 2S from McIntyre Group Ltd. Usually the amphoteric is used as a 40% active material product, with the above percentages reflecting active material.

The nonionic surfactants useful in the present invention are preferably formed from a fatty alcohol, a fatty acid, or a glyceride with a C₈ to C₂₄ carbon chain, preferably a C₁₂ to C₁₈ carbon chain, more preferably a C₁₆ to C₁₈ carbon chain, derivatized to yield a Hydrophilic-Lipophilic Balance (HLB) of at least 10. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 10. Preferably the nonionic surfactants contain ethoxylate in a molar content of from 10-25, more preferably from 10-20 moles.

Nonionic surfactants may be selected from, but are not limited to, the following:

| # of Cs | Name | Trade Name |
|---|---|---|
| C-12 | Laureth-23 | BRIJ 35, available from ICI Surfactants |
| C-16 | Ceteth-10 | BRIJ 56, available from ICI Surfactants |
| C-16 | Ceteth-20 | BRIJ 58, available from ICI Surfactants |
| C-16 | IsoCeteth-20 | Arlasolve 200, available from ICI Surfactants |
| C-18 | Steareth-10 | Volpo S-10, available from Croda Chemicals Ltd. |
| C-18 | Steareth-16 | Solulan-16, available from Amerchol Corp. |
| C-18 | Steareth-20 | BRIJ 78, available from ICI Surfactants |
| C-18 | Steareth-25 | Solulan-25, available from Amerchol Corp. |
| C-18 = | Oleth-10 | BRIJ 97, available from ICI Surfactants |
| C-18 = | Oleth-20 | Volpo-20, available from Croda Chemicals Ltd. |

Alkyl polyglucose surfactants sold under the name PLANTAREN, available from Cognis, may also be used. The nonionic surfactant is preferably present in an amount of from greater than 0 to 30% by weight, preferably 10 to 20% by weight, based on the weight of the composition.

In one preferred embodiment of the composition of the present invention, within the LAN in particular, the lecithin, the amphoteric surfactant, and the nonionic surfactant are present in the composition such that the nonionic surfactant and the amphoteric surfactant are each present in an amount by weight greater than the amount of lecithin. In a more preferred embodiment, the amount of lecithin in the composition is kept fixed while the amounts of the amphoteric and nonionic surfactants are increased. In a still more preferred embodiment, calculating the lecithin as present at a value of 1, the phospholipid, amphoteric surfactant and nonionic surfactant are preferably present in the composition in a ratio ranging from 1/1/1.8 and above by weight relative to the whole composition, i.e., where the amounts of the surfactants can be increased independently of each other but the amount of lecithin stays fixed. The ratio is considered to be “above” 1/1/1.8 when the amount of either of the surfactants increases. Another preferable range is from 1/1.2/2 and above. A further preferred ratio is 1/1.2/3 and above, and more preferably above 1/1.2/4. The loading capability for hydrophobes carried by the LAN system of the present invention is maximized if the ratio of nonionic surfactant to lecithin is minimized, with bilayers formed by the lecithin still being solubilized, because an excess of nonionic surfactant may disrupt the organized structure.

In one preferred embodiment, the composition of the present invention comprises ALCOLEC S (soy lecithin), MIRANOL C2M-SF Conc. (disodium cocamphodipropionate, an amphoteric surfactant), ARLASOLVE 200 (IsoCeteth-20, a nonionic surfactant) in a ratio of 5/6/10 (1:1.2:2) and 5/6/20 (1:1.2:4) wherein the ratios are calculated by weight relative to the whole composition. Typically, LAN compositions of the invention can resist storage at 45° C. for three months or more, which would predict that they have a shelf life at room temperature of at least three years.

Film forming polymers useful herein are non-neutralized or partially neutralized, preferably non-neutralized, polymers and resins, most preferably non-neutralized resins, wherein the polymers and resins include but are not limited to those containing carboxyl moieties, such as acrylates and other carboxy polymers. Typically, water-insoluble polymers and resins have to be neutralized to about 90% of their carboxyl moieties to make them water soluble for the purpose of formulating products in aqueous solution and for the purpose of making products which have good non-build-up properties, i.e., can be easily washed off the hair after use. However, when used with the compositions of the present invention complete, some (e.g., up to 90%) or no neutralization is needed to effectively use these polymers/resins. It is believed that the combination of the lecithin, the nonionic surfactant, and the amphoteric surfactant of the present invention provides the usefulness of the water-insoluble polymers or resins.

The following are examples of film forming polymers that can be incorporated into the compositions of the present invention. The list is not intended to be limiting:
AMPHOMER LV-71 from National Starch (octylacrylamide/acrylates/butylaminoethyl methacrylate copolymer),
OMNIREZ-2000 from ISP (PVM/MA half ethyl ester copolymer),
RESYN 28-2930 from National Starch (Vinyl acetate/crotonates/vinyl neodecanoate copolymer),
LUVIMER 100P from BASF (t-butyl acrylate/ethyl acrylate/methacrylic acid), and
ULTRAHOLD STRONG from BASF (acrylic acid/ethyl acrylate/t-butyl acrylamide).
Unneutralized or partially neutralized water-insoluble latexes can also be used as invention film-forming polymers. Included are the following latexes:
AMERHOLD DR-25 from Amerchol (acrylic acid/methacrylic acid/acrylates/methacrylates),
LUVIMER 36D from BASF (ethyl acrylate/t-butyl acrylate/methacrylic acid), and
ACUDYNE 258 from Rohm & Haas (acrylic acid/methacrylic acid/acrylates/methacrylates/hydroxy ester acrylates).

The film forming polymer is preferably present in amounts ranging from greater than 0 to 5% by weight, preferably from 0.001 to 3% by weight, and more preferably from 0.01 to 2% by weight, based on total weight of composition. The concentrations of LAN and film former can be adjusted by one of ordinary skill in view of this disclosure, as can neutralization extent.

Cationic polymers useful herein include polyquaternium 4, polyquaternium 6, polyquaternium 7, polyquaternium 10, polyquaternium 11, polyquaternium 16, polyquaternium 22, and polyquaternium 32. Cationic polymers useful in the present invention include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. Preferred cationic polymers include POLYMER JR-125, POLYMER JR-400, Polymer JR-30M hydroxyethyl cellulosic polymers (polyquaternium 10) available from AMERCHOL; JAGUAR C13-S, guar hydroxypropyltrimonium chloride, available from Meyhall; and MERQUAT 100 and 280, a dimethyl dialkyl ammonium chloride (polyquaternium 6) available from CALGON. The cationic polymer is preferably present in an amount of from greater than 0 to 5% by weight, preferably from 0.01 to 1% by weight, and more preferably from 0.1 to 0.5% by weight, based on the total weight of the inventive composition.

The invention compositions preferably also further comprise water, and can in addition contain ingredients such as silicones, anionic surfactants, organic salts, inorganic salts, proteins, hair dyes, water-soluble polymers, quaternary ammonium compounds, complex and simple carbohydrates, amino acids, preservatives and fragrances.

It has surprisingly been discovered that in addition to conditioning, frizz control and curl retention benefits, volumizing and bodifying benefits can also be realized by optimizing the amounts of film-forming and cationic polymers present in the composition, as well as the composition's pH. More particularly, by employing the composition of the present invention at a pH of from 5 to 12, preferably from 5 to 10, and more preferably from 6 to 8, volumizing/bodifying benefits are obtained. The film-forming polymer and cationic polymer will typically be employed in ratio by weights ranging from 500:1 to 0.5:1, preferably 10:1 and more preferably 2:1.

Another embodiment of the present invention is drawn to a process for preparing the invention compositions. This process preferably comprises: (a) combining the following ingredients to obtain a mixture: at least one lecithin, at least one nonionic surfactant, and at least one amphoteric surfactant, where the nonionic surfactant and amphoteric surfactant are each present in an amount by weight equal to or greater than the amount of the lecithin, (b) heating the mixture obtained in step (a), and (c) adding an aqueous solution to the heated mixture to obtain the desired carrier system. Water-insoluble ingredients may be added in step (a). The mixture is preferably heated at a temperature of 65° C. to 85° C., depending on the melting points of the solid surfactants.

More specifically, the preparation of the LAN system of the present invention may preferably be carried out as follows. Lecithin (L) is dispersed in water. The water-insoluble material is combined with nonionic surfactant(s) (N) at appropriate ratios and added to the lecithin/water dispersion. An amphoteric surfactant (A) is added and the mixture is heated, preferably to a temperature of from 75° C. to 85° C. The combination of these ingredients results in a solution which is clear to slightly hazy and is referred to as the “LAN,” which can then be used as a “raw material” to make finished products.

Alternatively, lecithin, amphoteric surfactant(s) and nonionic surfactant(s) can be weighed to appropriate ratios and heated to 70° C. with stirring. Water is then added q.s. at the same temperature. Another alternative method of preparation comprises adding the water-insoluble ingredient with mixing after solutions have cooled. This last alternative method helps protect heat-sensitive water-insoluble ingredients.

The resulting compositions may vary from clear to slightly hazy and are infinitely dilutable with water. The slight haze can be overcome by adjusting the ratio of lecithin to the surfactants or reducing concentrations of water-insoluble ingredients.

Once the LAN is prepared the film forming polymer and cationic polymer can be added thereto and stirred at from, e.g., 50° C. to 85° C., until homogeneous if desired.

Adjusting the pH of the composition to the above-disclosed optimized levels can be accomplished using conventional techniques known by those skilled in the art of formulation.

With respect to hair compositions, the present invention compositions can take any form, including hair products, e.g., for normal hair, color-treated hair, dry hair, fine hair, and damaged hair. For each type of hair, the composition can be used to create a regimen comprising shampoo, conditioner, styling and deep treatment, (i.e., deep conditioner). Compositions for these products preferably contain lecithin (L), at least one amphoteric surfactant (A), such as disodium cocoamphodipropionate, and at least one nonionic surfactant (N), e.g., a blend of Oleth-10 and PPG-5-Ceteth-20 as LAN. Additional nonionic, amphoteric, and also anionic surfactants can be added. The compositions may further contain at least one water-insoluble ingredient (also referred to as a hydrophobe) such as olive, mineral, or other oils, octyl salicylate, Vitamin E (Tocopherol), octyl methoxycinnamate, and ceramides including 2-oleamido-1,3-octadecanediol.

In general, the concentration of the LAN may be increased within each regimen from shampoo to conditioner to deep treatment. Thus, the deep treatment formulations preferably have the most concentrated hydrophobe-carrying LAN.

The compositions of the invention can further comprise proteins including hydrolyzed soy protein, lauryldimonium hydrolyzed soy protein (cationic Soya protein) and wheat amino acids. The proteins can also include corn, wheat, milk, or silk proteins, collagens, keratins, or others. Furthermore, taurine and arginine hydrochloride may be associated therein to maximize protein binding to the hair

Further, shampoos, conditioners, styling compositions and deep treatments within the scope of the present invention may be used on hair which has been treated, e.g., with color (dye or bleach) or chemicals (permanent wave or straightening), or which is dry or fine and show significant substantivity for the hair. Invention compositions preferably take the following forms: shampoo, gel, cream, foam, pomades, aerosol, spray, etc.

Further descriptions of components useful herein, including the LAN, cationic polymers, and film forming polymers, can be found in U.S. Pat. Nos. 6,015,574, 6,440,456, 6,436,436 and 6,221,389, 6,558,697 and 6,524,614, all of which are incorporated herein by reference.

The present invention will be better understood by the examples which follow, all of which are intended for illustrative purposes only, and are not meant to unduly limit the scope of the invention in any way.

### EXAMPLES

Example 1 shampoo in accordance with the present invention:

| | |
|---|---|
| SODIUM CHLORIDE | 1.0 |
| CITRIC ACID | 1.0 |
| BLUE 1 | 0.00001 |
| LECITHIN | 0.1 |
| 2-OLEAMIDO-1,3-OCTADECANEDIOL | 0.01 |
| FRAGRANCE | 0.5 |
| POLYQUATERNIUM-10 | 0.2 |
| OCTYLACRYLAMIDE/ACRYLATES/ | 0.4 |
| BUTYLAMINOETHYL METHACRYLATE COPOLYMER | |
| DMDM HYDANTOIN | 0.25 |
| METHYLPARABEN | 0.3 |
| WATER | 66.13999 |
| COCAMIDOPROPYL BETAINE | 5 |
| PPG-5-CETETH-20 | 3.1 |
| COCAMIDE MIPA | 3 |
| SODIUM LAURETH SULFATE | 15 |
| DISODIUM COCOAMPHODIPROPIONATE | 4.5 |
| WHEAT AMINO ACIDS (and) HYDROLYZED SOY | 0.5 |
| PROTEIN | |

Comparative shampoo:

| | |
|---|---|
| SODIUM CHLORIDE | 1.0 |
| CITRIC ACID | 1.0 |
| BLUE 1 | 0.00001 |
| LECITHIN | 0.0 |
| 2-OLEAMIDO-1,3-OCTADECANEDIOL | 0.01 |
| FRAGRANCE | 0.50 |
| POLYQUATERNIUM-10 | 0.00 |
| OCTYLACRYLAMIDE/ACRYLATES/ | 0.00 |
| BUTYLAMINOETHYL METHACRYLATE COPOLYMER | |
| DMDM HYDANTOIN | 0.25 |
| METHYLPARABEN | 0.30 |
| WATER | 76.913999 |
| COCAMIDOPROPYL BETAINE | 0.00 |
| PPG-5-CETETH-20 | 0.00 |
| COCAMIDE MIPA | 3.00 |
| SODIUM LAURETH SULFATE | 15.00 |
| DISODIUM COCOAMPHODIPROPIONATE | 4.50 |
| WHEAT AMINO ACIDS (and) HYDROLYZED SOY | 0.50 |
| PROTEIN | |

Assessments were done in the salon with trained stylists (on 12 panelists). The example 1 formulation having a pH of 7.5 was tested versus the comparative formulation having a pH of 4.5. The results from the half-head shampoo test showed the shampoo in accordance with the present invention increased volume and body. The following table illustrates the improvements shown with the shampoo of example 1:

| | |
|---|---|
| shampoo | statistically |

| Attributes | comparative | example 1 | significant? |
|---|---|---|---|
| Root lift | 2.06 | 2.33 | yes |
| Body | 2.03 | 2.75 | yes |
| Volume | 2.00 | 2.91 | yes |
| Light hair | 2.13 | 2.79 | yes |
| Overall preference | 2.00 | 2.58 | yes |

A preferred embodiment of the invention that one of ordinary skill in the art is now able to make and use in view of this invention description is a composition comprising at least one lecithin, at least one amphoteric surfactant, at least one nonionic surfactant, at least one film forming polymer, and at least one cationic polymer. A highly preferred embodiment of the invention is a composition, having a pH of from 6 to 8, comprising from 0.05 to 0.5 wt. % of at least one lecithin, from 1 to 3 wt. % of at least one amphoteric surfactant, from 2 to 4 wt. % of at least one nonionic surfactant, from 0.1 to 2 wt. % of at least one film forming polymer, and from 0.2 to 0.6 wt. % of at least one cationic polymer, wherein said wt. % s are based on total weight of said composition, these compositions optionally further comprising water, preferably 1-90 wt % water based on the total weight of the composition. Fine or thin hair treated with the above-disclosed composition experiences a volumizing/bodifying effect together with conditioning, frizz control and curl retention benefits.

All references, texts, patents, patent applications, product literature, product brochures and MSDS sheets, documents, publications etc., mentioned above are incorporated herein by reference. Where a numeral range or limit is described, all values therewithin are specifically included as if separately written out.

1. An aqueous composition comprising at least one lecithin, at least one amphoteric surfactant, at least one nonionic surfactant, at least one film forming polymer, and at least one cationic polymer, and wherein the composition has a pH of from about 5 to about 12.

2. The composition of claim 1, wherein the composition has a pH of from about 6 to about 8.

3. The composition of claim 1, wherein the at least one film forming polymer and the at least one cationic polymer are present in the composition in a ratio by weight of about 500:1, respectively.

4. The composition of claim 1, wherein the at least one film forming polymer and the at least one cationic polymer are present in the composition in a ratio by weight of about 2:1, respectively.

5. The composition of claim 1, comprising from greater than 0 to about 10 wt. % of at least one lecithin, from greater than 0 to about 30 wt. % of at least one amphoteric surfactant, from greater than 0 to about 30 wt. % of at least one nonionic surfactant, from greater than 0 to 5 wt. % of at least one film forming polymer, and from greater than 0 to 5 wt. % of at least one cationic polymer, wherein said wt. % s are based on total weight of said composition.

6. The composition of claim 1, wherein the water is present in an amount of from about 1 to about 90 wt % based on the total weight of the composition.

7. The composition of claim 1, wherein the at least one film forming polymer and the at least one cationic polymer are present in the composition in a ratio by weight of about 0.5:1, respectively.

8. The composition of claim 5, wherein the water is present in an amount of from about 1 to about 90 wt % based on the total weight of the composition.

9. The composition of claim 1, wherein the composition is a shampoo.

10. The composition of claim 1, wherein the composition is a conditioner.

11. The composition of claim 1, wherein the composition is a styling composition.

12. The composition of claim 1, wherein the composition is a deep treatment composition

13. A method for volumizing hair, comprising contacting the hair with the composition of claim 1.

14. The method of claim 13, wherein the hair is fine or thin.

15. The method of claim 13, wherein the hair is permed.

16. A method for simultaneously adding volume and maintaining the shape and/or curl definition of hair, comprising contacting hair having a shape and/or curl definition with the composition of claim 1.
